SEQUENCE LISTING (10 sequences)

SEQ ID NO: 1 - DNA, 24 bp, Homo sapiens
agaaagtgcacgaagaggagatcg

SEQ ID NO: 2 - DNA, 24 bp, Homo sapiens
atcttcacgttgaggaggtcttgg

SEQ ID NO: 3 - DNA, 23 bp, Homo sapiens
tggaaacttccaagacctgactc

SEQ ID NO: 4 - DNA, 24 bp, Homo sapiens
cagagataaaaccctgcctcctga

SEQ ID NO: 5 - DNA, 24 bp, Homo sapiens
agacactgttcccaaagccttgac

SEQ ID NO: 6 - DNA, 24 bp, Homo sapiens
accccacatttccttctccttctc

SEQ ID NO: 7 - DNA, 20 bp, Homo sapiens
agccacatcgctcagacacc

SEQ ID NO: 8 - DNA, 20 bp, Homo sapiens
gtactcagcggccagcatcg

SEQ ID NO: 9 - DNA, 25 bp, Homo sapiens
cttgctgcagaagtgggtggaggaa

SEQ ID NO: 10 - DNA, 21 bp, Homo sapiens
ctgcagtgtgggtttcgggca

(19) United States
(12) Patent Application Publication
(10) Pub. No.: US 2013/0295637 A1
(43) Pub. Date: Nov. 7, 2013

(54) FIBROUS SUBSTRATES FOR CELL PROPAGATION AND DIFFERENTIATION

(71) Applicants: Hongfang Lu, Singapore (SG); Andrew Chwee Aun Wan, Singapore (SG); Jackie Y. Ying, Singapore (SG); Karthikeyan Narayanan, Singapore (SG)

(72) Inventors: Hongfang Lu, Singapore (SG); Andrew Chwee Aun Wan, Singapore (SG); Jackie Y. Ying, Singapore (SG); Karthikeyan Narayanan, Singapore (SG)

(21) Appl. No.: 13/814,501

(22) PCT Filed: Aug. 5, 2011
(86) PCT No.: PCT/SG11/00275

§ 371 Date: May 14, 2013

(30) Foreign Application Priority Data
Aug. 5, 2010 (SG) 201005751-1

(51) Int. Cl. C12N 11/04

(52) U.S. Cl. CPC C12N 11/04
USPC 435/178

(57) ABSTRACT

The present invention relates to a method of releasably encapsulating pluripotent embryonic stem cells in a degradable continuous polyionic fiber for tissue culture, wherein the encapsulated embryonic stem cells are able to maintain a pluripotent phenotype in tissue culture; the method comprising (a) contacting an aqueous solution of a polyanion with an aqueous solution of a polycation to form an interface between the aqueous solution of polyanion and the aqueous solution of polycation, and wherein the aqueous solution of polyanion or the aqueous solution of polycation or both the aqueous solution of polyanion and the aqueous solution of polycation comprises a suspension of pluripotent embryonic stem cells; (b) drawing a continuous polyionic fiber which comprises encapsulated pluripotent embryonic stem cells from the interface; (c) passing the continuous polyionic fiber comprising encapsulated pluripotent embryonic stem cells in a continuous process through a solution which reduces secondary complexation of the components of the polyionic fiber.

### INCORPORATION BY REFERENCE

This application claims priority from Singapore patent application no. 201005751-1 filed on 5 Aug. 2010, the entire contents of which are incorporated herein by reference in their entirety.

### TECHNICAL FIELD

The invention relates generally to the field of cell culture. More specifically, the invention relates to constructs for culturing stem cells and methods for their production.

### BACKGROUND

Human embryonic stem cells (hESCs) are pluripotent cells derived from the inner cell mass of embryonic blastocysts. A large number of studies have shown the ability of hESCs to differentiate into a variety of lineages, including insulin-producing cells, neural precursor cells and cardiomyocytes, showing great promise that these cells will be used as a renewable source to treat a number of human ailments, including type 1 diabetes, Parkinson's and cardiovascular disease.

Despite their tremendous therapeutic potential, realization of the clinical application of hESCs and their derivatives relies on producing therapeutic quantities of cells with sufficient quality for transplantation into patients. In attempting to achieve these goals, research to date has been focused on manipulating hESC culture techniques to provide scalable hESC expansion and controlled differentiation. In comparison to conventional culture of hESCs on human/mouse embryonic fibroblast feeder layers (MEFs) and conditioned media, ECM-coated surfaces and chemically defined media have the potential to provide safer and more reproducible substrate and soluble medium components for hESC culture. However, the practical limit for large-scale production and lack of control over spontaneous stem cell differentiation are issues that are yet to be resolved.

During embryogenesis, hESCs are embedded in a three-dimensional (3D) environmental milieu which regulates cell proliferation and differentiation. 3D culture may imitate the in vivo microenvironment by enhancing cell-cell and cell-matrix interactions and subsequent cell signaling. In this case, 3D aggregates of hESCs produced using the embryonic body (EB) culture technique, which more accurately recapitulates early development processes, have been widely utilized with the aim of hESC culture and differentiation. While the EB suspension culture may exhibit potential for a scalable platform, unresolved difficulties still exist including heterogeneous sizes leading to variations in the differentiation lineage, and premature attachment to the culture substrate leading to loss of EBs from suspension. In an attempt to address these issues, a variety of 3D hydrogel scaffolds with the appropriate chemical and physical factors have been developed and adopted for modulating the self-renewal and differentiation of hESCs. However, difficulties exist in effectively harvesting the cultured hESCs and derivatives from the hydrogel materials.

In view of these and other deficiencies in currently existing techniques, there is a need for new systems and methods for culturing stem cells such as hESCs.

### SUMMARY OF THE INVENTION

The present invention overcomes one or more of deficiencies in the prior art by providing an engineered 3D fibrous encapsulation culture system capable of supporting the large-scale expansion, isolation and/or controlled differentiation of stem cells.

In a first aspect, the invention provides a method of releasably encapsulating pluripotent embryonic stem cells in a degradable continuous polyionic fiber for culture, wherein the encapsulated embryonic stem cells are able to maintain a pluripotent phenotype in tissue culture; the method comprising

(a) contacting an aqueous solution of a polyanion with an aqueous solution of a polycation to form an interface between the aqueous solution of polyanion and the aqueous solution of polycation, and wherein the aqueous solution of polyanion or the aqueous solution of polycation or both the aqueous solution of polyanion and the aqueous solution of polycation comprises a suspension of pluripotent embryonic stem cells;

(b) drawing a continuous polyionic fiber which comprises encapsulated pluripotent embryonic stem cells from the interface;

(c) passing the continuous polyionic fiber comprising encapsulated pluripotent embryonic stem cells in a continuous process through a solution which reduces secondary complexation of the components of the polyionic fiber.

In a second aspect, the invention provides a method of culturing pluripotent embryonic stem cells, comprising releasably encapsulating pluripotent embryonic stem cells in a degradable continuous polyionic fiber and culturing the encapsulated pluripotent embryonic stem cells.

In a third aspect, the invention provides a method of releasing encapsulated cells from a continuous polyionic fiber which comprises chitin and alginate, the method comprising exposing the polyionic fiber comprising encapsulated cells to chitinase or to chitinase and alginate lyase simultaneously and/or sequentially to degrade the polyionic fiber sufficiently to allow the release of the encapsulated cells from the polyionic fiber.

In a fourth aspect, the invention provides a method of cryopreserving releasably encapsulated pluripotent embryonic stem cells, the method comprising:

(i) encapsulating the pluripotent embryonic stem cells in a degradable continuous polyionic fiber by
(a) contacting an aqueous solution of a polyanion with an aqueous solution of a polycation to form an interface between the aqueous solution of polyanion and the aqueous solution of polycation, and wherein the aqueous solution of polyanion or the aqueous solution of polycation or both the aqueous solution of polyanion and the aqueous solution of polycation comprises a suspension of pluripotent embryonic stem cells;
(b) drawing a continuous polyionic fiber which comprises encapsulated pluripotent embryonic stem cells from the interface; and
(c) passing the continuous polyionic fiber comprising encapsulated pluripotent embryonic stem cells in a continuous process through a solution which reduces secondary complexation of the components of the polyionic fiber; and then

(ii) cryopreserving the encapsulated pluripotent embryonic stem cells.

In a fifth aspect, the invention provides an assembly for maintaining pluripotent embryonic stem cells in culture, comprising a degradable continuous polyionic fiber comprising releasably encapsulated pluripotent embryonic stem cells, wherein the polyionic fiber is held by a fiber holder, and wherein the assembly is of neutral buoyancy or is submersible in culture medium.

In a sixth aspect, the invention provides an assembly for maintaining pluripotent embryonic stem cells in culture, comprising a polyionic fiber comprising releasably encapsulated pluripotent embryonic stem cells, wherein the polyionic fiber is held by a fiber holder, and wherein the assembly is of neutral buoyancy or is submersible in culture medium.

In one embodiment of the fifth or sixth aspect, the polyionic fiber is formed from a polyanionic solution comprising alginate and a polycationic solution comprising chitin and/or chitosan.

In one embodiment of the fifth or sixth aspect, the assembly is neutrally bouyant in the culture medium.

In a seventh aspect, the invention provides a method of releasably encapsulating pluripotent embryonic stem cells in a degradable polyionic fiber for culture, wherein the encapsulated embryonic stem cells are able to maintain a pluripotent phenotype in tissue culture; the method comprising

(a) contacting an aqueous solution of a polyanion with an aqueous solution of a polycation to form an interface between the aqueous solution of polyanion and the aqueous solution of polycation, and wherein the aqueous solution of polyanion or the aqueous solution of polycation or both the aqueous solution of polyanion and the aqueous solution of polycation comprises a suspension of pluripotent embryonic stem cells;

(b) drawing a polyionic fiber which comprises encapsulated pluripotent embryonic stem cells from the interface;

(c) passing the polyionic fiber comprising encapsulated pluripotent embryonic stem cells in a process through a solution which reduces secondary complexation of the components of the polyionic fiber.

In one embodiment of the sixth or seventh aspect, the fiber is a discontinous fiber.

In one embodiment of the sixth or seventh aspect, the fiber is packed in a column or cartridge.

In one embodiment of the first, second, third, fourth, fifth, sixth or seventh aspect, the cells are human pluripotent embryonic stem cells.

In one embodiment of the first, second, third, fourth, fifth, sixth or seventh aspect, the cells are induced pluripotent stem cells, cancer cells, or cancer stem cells.

In one embodiment of the first, fourth or seventh aspect, the solution of polycation and/or the solution of polyanion comprises between about 10 million cells/ml and about 100 million cells/ml.

In one embodiment of the first, second, third, fourth, or seventh aspect, the fiber is mounted on a fiber holder.

In one embodiment of the first, second, third, fourth, or seventh aspect, the fiber holder with mounted fiber is neutrally bouyant in a culture medium.

In one embodiment of the first, fourth or seventh aspect, the polycationic solution comprises chitin and has a viscosity of between about 20 cps and about 200 cps.

In one embodiment of the first, fourth or seventh aspect, the polyanionic solution comprises alginate and has a viscosity of between about 200 cps and 300 cps.

In one embodiment of the first, fourth or seventh aspect, the fiber is drawn at a rate of between about 5 mm and about 10 mm/second.

In another embodiment of the first, fourth or seventh aspect, the polyanion is alginate.

In an additional embodiment of the first, fourth or seventh aspect, the polycation is chitin or chitosan.

In a further embodiment of the first, fourth or seventh aspect, the solution which reduces secondary complexation of the components of the polyionic fiber comprises CaCl₂.

In a further embodiment of the second, third, fifth or sixth aspect, the polyionic fiber is prepared by the method of the first or seventh aspect.

In another embodiment of the first, fourth, fifth, sixth or seventh aspect, the chitin is a water soluble chitin with a degree of deacetylation of approximately 50%.

### BRIEF DESCRIPTION OF THE DRAWINGS

Preferred embodiments of the invention will now be described, by way of example is only, with reference to the accompanying figures wherein:

FIG. 1 provides a schematic diagram of the fabrication of hESCs-microfiber constructs.

FIG. 2 provides microscopy images (A-D) and a graph (E) showing morphology or proliferation of encapsulated hESCs at various culturing time points (A-C) with hESCs grown on matrigel layer as controls (D). hESCs formed small cell clusters after encapsulation and proliferated with time in culture. Gross morphology of encapsulated cells under light (Ai, Bi, Ci and Di) and fluorescent (Aii, Bii, Cii and Dii) microscopes. (E) Proliferation profile of encapsulated hESCs as a function of time. Encapsulated cells were released by enzyme decapsulation method and counted under microscope.

FIG. 3 provides graphs (A-C) and microscopy images (D-K) based on hESCs released from microfibers using enzyme method. Cytotoxicity assays of chitinase (A), alginate lyase (B) and a mixture of chitinase and alginate lyase (C) are shown. The encapsulated cell-fiber constructs (D, E) were treated with chitinase-alginate lyase at 37° C. for 5 mins, the fibers were decomposed (F,G), and cell clusters collected by centrifugation (H,I). The released cell clusters attached to the MEF layers (J,K).

FIG. 4 provides flow cytometry histogram plots (A, B), a graph (C) and light microscopy images (D) arising from an in vitro assessment of the undifferentiated state of released hESC clusters from alginate chitin fibers (as assessed by flow cytometry, and gene and immunological detection of Oct4 and Nanog). Flow cytometry study for GFP-positive hESCs grown (A) on Matrigel layers and (B) in chitin-alginate fibers. (C) RT-PCR results of gene expression of Oct4 and Nanog from hESCs cultured in the fiber, MEF and Matrigel layers. (D) Immunostaining of stem cell marker Nanog in released hESC cluster.

FIG. 5 provides (A) microscopy images from histological morphologic analyses, and (B) a pictorial representation of gene expression of hESC derived teratomas 8 weeks after implantation in SCID mice. Teratomas derived from these cells consisted of highly differentiated cells and tissues derived from all three germ layers. Representative sections stained with hematoxylin and eosin (H&E) stain (A). A pictorial comparison of changed genes detected by PCR array together with functional cluster analysis (B). Different shades represent average gene expression changes (teratoma/control hESCs) relative to the median with dark shading and light shading representing an increase or decrease in fold expression, respectively. “S1” and “S2” represent individual teratoma from different mice.

FIG. 6 provides microscopy images (A-C) and a photograph of a gel indicative of the direct differentiation of hESC clusters into neuron tissue-like structures in chitin-alginate fibers. H9 hESCs, propagated in chitin alginate fibers for 1 week, were incubated with neuron stimulation and differentiation medium for 20 days. (A-C) Immunological staining of neuron filament in released clusters 48-hr postseeding on laminin coated plate. (B) The edge of the aggregate. (C) The center of the aggregate. (D) Neuron specific genes were expressed in the differentiated cell clusters, as revealed by PCR. “hESC”: undifferentiated embryonic stem cells; “D-neuron”: neuron cells differentiated from hESC; “SH-Sy5y”: neuroblastoma cell line; “NT”: negative control.

FIG. 7 provides microscopy images showing morphology and proliferation of encapsulated hESCs after cryopreservation. Six-day-culture hESC-fiber constructs before cryopreservation (A), 2-day culture (B) and 4-day culture (C) after being taken from cryopreservation. On the fifth day, the hESC aggregates were released from fibers by chitinase-alginate lyase enzymes (D&E) and subcultured on MEF layer on a culture plate (F&G) and Matrigel-coated culture plate (H&I). Gross morphology of cells under light (D, F & H) and fluorescent (A, B, C, E, G & I) microscopes.

### DEFINITIONS

As used in this application, the singular form “a”, “an” and “the” include plural references unless the context clearly dictates otherwise. For example, the term “a polymer” also includes a plurality of polymers.

As used herein, the term “comprising” means “including.” Variations of the word “comprising”, such as “comprise” and “comprises,” have correspondingly varied meanings. Thus, for example, a construct “comprising” a given type of polymer may consist exclusively of that type of polymer or may include one or more additional types of polymers.

It will be understood that use of the term “about” herein in reference to a recited numerical value includes the recited numerical value and numerical values within plus or minus ten percent of the recited value.

It will be understood that use of the term “between” herein when referring to a range of numerical values encompasses the numerical values at each endpoint of the range. For example, a polymer of between 10 monomers and 20 monomers in length is inclusive of a polymer of 10 monomers in length and a polymer of 20 monomers in length.

As used herein, the term “hydrogel” refers to a hydrophilic polymeric network capable of absorbing water without dissolving (i.e. a water insoluble, water-containing material).

As used herein, the term “polyelectrolyte” refers to a polymer in which the repeating units bear an electrolyte group that dissociates in aqueous solution causing the polymer to become charged.

Reference herein to a component that is “encapsulated” in a fiber of the present invention will be understood to mean that the component is unable move out of the fiber whilst the integrity of the fiber is maintained.

Reference herein to a fiber that is “degradable” will be understood to mean that the fiber has a potential to be modified in a manner facilitating release of encapsulated components.

Reference herein to a component (e.g. a cell) that is “releasably encapsulated” will be understood to mean that the component is encapsulated in a fiber that is capable of being modified in a manner facilitating release of the encapsulated component.

Any description of prior art documents herein, or statements herein derived from or based on those documents, is not an admission that the documents or derived statements are part of the common general knowledge of the relevant art.

For the purposes of description all documents referred to herein are hereby incorporated by reference in their entirety unless otherwise stated.

### DETAILED DESCRIPTION

Despite the significant therapeutic potential of stem cells in a wide range of clinical applications, progress has been hindered due to the inability to provide for large-scale stem cell production and/or a lack of control over stem cell differentiation. Furthermore, difficulties exist in effectively harvesting cultured stem cells and their derivatives from various culture systems.

The present invention provides fibrous constructs for the effective propagation of stem cells. The constructs are scalable in the sense that they may be expanded in size to accommodate the propagation of large numbers of stem cells. Stem cells propagated in the constructs may be maintained in an undifferentiated state, or alternatively induced to differentiate into specific lineages within the constructs. The constructs may be used for the directed differentiation and organization of stem cells into tissue-like structures. Constructs of the present invention may be degradable in the sense that they may be decomposed facilitating the release of propagated and/or differentiated cells.

Also provided are methods for production of the fibrous constructs. In general, the methods comprise encapsulating stem cells within one or more polyionic fibers generated from a degradable material. Fibrous constructs so produced may be used for the propagation and/or differentiation of stem cells, and then degraded to allow release of the cells.

Methods for cryopreservation of the fibrous constructs are also provided herein allowing the constructs, which may optionally comprise stem cells, to be stored for subsequent thawing and use. In general, the cryopreservation methods have minimal adverse impacts on the integrity and functional capacity of the constructs and/or cells encapsulated within the constructs.

### Polyionic Fibers

The present invention provides fiber constructs in which stem cells can be encapsulated, and propagated and/or differentiated.

A fiber according to the present invention may comprise a matrix encapsulating one or more desired components. The air-liquid interface of the fiber may provide a membrane-like structure or “skin” forming the external surface of the fiber.

The matrix may be porous supporting the migration and/or self assembly of components encapsulated within it.

Although any suitable matrix may be used, the matrix is preferably polymer-based and aqueous.

In certain embodiments, the fiber matrix may be a hydrogel. As used herein, the term “hydrogel” refers to a hydrophilic polymeric network capable of absorbing water without dissolving (i.e. a water insoluble, water-containing material).

Suitable hydrogels include macromolecular and polymeric materials into which water and other small molecules (e.g. biologics such as extracellular matrix proteins and drugs) can easily diffuse. Non-limiting examples include hydrogels prepared by cross-linking of both natural and synthetic hydrophilic polymers via ionic, covalent, and/or hydrophobic bonds introduced by chemical cross-linking agents and/or electromagnetic radiation (e.g. ultraviolet light). For example, suitable hydrogels include those prepared by cross-linking of poly(vinyl pyrrolidone); polysaccharides (e.g. hyaluronic acid, chondroitin sulfate, dextran, alginate, heparin or heparin sulfate); poly(vinyl alcohol); polyethers (e.g. polyakyleneoxides including poly(ethylene oxide), poly(ethylene glycol), poly(ethylene oxide)-co-(poly(propyleneoxide) block copolymers); or proteins (e.g. albumin, ovalbumin, gelatin, polyamino acids or collagen).

The hydrogel matrix may be provided in any suitable configuration. For example, the hydrogel may be in the form of sheets, particles, rods, beads, or irregular shapes.

The hydrogel matrix may be natural or synthetic. Non-limiting examples of suitable hydrogels composed of synthetic polymers include polyhydroxy ethyl methacrylate, and chemically or physically cross-linked polyacrylamide, poly(N-vinyl pyrolidone), polyvinyl alcohol, polyethylene oxide, and hydrolysed polyacrylonitrile. Non-limiting examples of suitable hydrogels composed of organic polymer hydrogels include covalent or ionically cross-linked polysaccharide-based hydrogels such as the polyvalent metal salts of alginate, pectin, heparin, carboxymethyl cellulose, hyaluronate and hydrogels from gellan, pullulan, chitin, chitosan, and xanthan.

In preferred embodiments, fibers of the invention may be fabricated (i.e. formed) by drawing up from an interface formed between two oppositely charged polyelectrolyte solutions. As known to those skilled in the field, a “polyelectrolyte” is a polymer in which the repeating units bear an electrolyte group that dissociates in aqueous solution causing the polymer to become charged.

Non-limiting examples of cationic polyelectrolytes that may be used in the formation of fibers of the invention include, but are not limited to chitin, chitosan, basic keratins, poly(lysine), polyglutamic acid, polyornithine, polyethyleneimine; galactosylated compounds of chitin, collagen, chitosan and methylated collagen; natural and synthetic carbohydrates; polypeptide polymers having a net positive charge; or combinations thereof.

Non-limiting examples of anionic polyelectrolytes that may be used in the formation of fibers of the invention include, but are not limited to, alginate; acidic keratins; gelatine; gellan; chondroitin sulphate; hyaluronic acid; fibrinogen; heparin; terpolymer consisting of methyl methacrylate, hydroxyethyl methacrylate and methacrylic acid; carboxymethylated, phosphorylated and/or sulfated derivatives, which include those of cellulose; deoxyribonucleic acid (DNA), ribonucleic acid (RNA) and their derivatives; natural and synthetic carbohydrate; polypeptide polymers having a net negative charge; or combinations thereof.

In certain embodiments, the fiber may be fabricated (i.e. formed) by drawing it from an interface formed between a polycationic solution comprising water soluble chitin and a polyanionic solution comprising alginate. The fiber may be a continuous fiber or a discontinuous fiber (e.g. packed in a column or cartridge).

Preferably, the fiber is a continuous fiber.

Fibers of the present invention may be porous. The porosity of the fibers (e.g. a hydrogel matrix) is generally of a size that allows the migration of components (e.g. cells, proteins, growth factors, nutrients, cellular wastes) within and into/out of the fiber.

In certain embodiments, the pore size of the fiber is between about 1 nanometer and about 20 micrometers. In other embodiments, the pore size of the matrix is less than about 20, 19, 18, 17, 16, 15, 14, 13, 12, 11, 10, 9, 8, 7, 6, 5, 4, 3, 2 or 1 micrometers. The pore size of the matrix may be between about 1 nanometer and 1000 nanometers (i.e. 1 micrometer), between about 10 nanometers and 1000 nanometers, between about 10 nanometers and about 500 nanometers, between about 100 nanometers and about 500 nanometers, or between about 10 nanometers and about 100 nanometers.

Without imposing any particular restriction or limitation, the diameter of a fiber of the present invention may be between about 1 micrometer and about 500 micrometers, between about 5 micrometers and about 500 micrometers, between about 10 micrometers and about 500 micrometers, between about 10 micrometers and about 400 micrometers, between about 10 micrometers and about 300 micrometers, between about 10 micrometers and about 200 micrometers, between about 50 micrometers and about 200 micrometers, between about 10 micrometers and about 100 micrometers, between about 10 micrometers and about 80 micrometers, between about 2 micrometers and about 100 micrometers, or between about 2 micrometers and about 50 micrometers.

Again without imposing any particular restriction or limitation, the length of a fiber may be in the range of about 0.1 cm to about 50 cm. For example, the length may be between about 1 cm and about 50 cm, between about 10 cm and about 50 cm, between about 10 cm and about 40 cm, or between about 1 cm and about 40 cm. In one embodiment, the length of the fiber is greater than about 10 cm.

### Encapsulated Components

Fibers according to the present invention may comprise encapsulated component(s).

A component that is “encapsulated” in a fiber of the present invention will generally be unable to move out of the fiber while the integrity of the fiber is maintained. However, as discussed in the sections below encapsulated components may be released from a fiber of the present invention by modifying the fiber in a manner that disrupts its integrity allowing release of the components. Accordingly, components may be “releasably encapsulated” in a fiber. It will be understood that despite an inability to move out of an intact fiber, an encapsulated component may potentially be capable of migrating within the fiber, although this is not necessarily a requirement.

In certain embodiments, the fibers may comprise encapsulated cells.

Non-limiting examples of cell types that may be encapsulated in fibers of the present invention include stem cells; embryonic stem cells; induced pluripotent stem cells; cancer cells; cancer stem cells; adult stem cells; blast cells; cloned cells; placental cells; keratinocytes; basal epidermal cells; urinary epithelial cells; salivary gland cells; mucous cells; serous cells; von Ebner's gland cells; mammary gland cells; lacrimal gland cells; ceruminpus gland cells; eccrine sweat gland cells; apocrine sweat gland cells; MpH gland cells; sebaceous gland cells; Bowman's gland cells; Brunner's gland cells; seminal vesicle cells; prostate gland cells; bulbourethral gland cells; Bartholin's gland cells; Littre gland cells; uterine endometrial cells; goblet cells of the respiratory or digestive tracts; mucous cells of the stomach; zymogenic cells of the gastric gland; oxyntic cells of the gastric gland; insulin-producing P cells; glucagon-producing a cells; somatostatin-producing DELTA cells; pancreatic polypeptide-producing cells; pancreatic ductal cells; Paneth cells of the small intestine; type II pneumocytes of the lung; Clara cells of the lung; anterior pituitary cells; intermediate pituitary cells; posterior pituitary cells; hormone secreting cells of the gut or respiratory tract; thyroid gland cells; parathyroid gland cells; adrenal gland cells; gonad cells; juxtaglomerular cells of the kidney; macula densa cells of the kidney; peri polar cells of the kidney; mesangial cells of the kidney; brush border cells of the intestine; striated ducted cells of exocrine glands; gall bladder epithelial cells; brush border cells of the proximal tubule of the kidney; distal tubule cells of the kidney; conciliated cells of the ductulus efferens; epididymal principal cells; epididymal basal cells; hepatocytes; fat cells; type I pneumocytes; pancreatic duct cells; nonstriated duct cells of the sweat gland; nonstriated duct cells of the salivary gland; nonstriated duct cells of the mammary gland; parietal cells of the kidney glomerulus; podocytes of the kidney glomerulus; cells of the thin segment of the loop of Henle; collecting duct cells; duct cells of the seminal vesicle; duct cells of the prostate gland; vascular endothelial cells; synovial cells; serosal cells; squamous cells lining the perilymphatic space of the ear; cells lining the endolymphatic space of the ear; choroid plexus cells; squamous cells of the pia-arachnoid; ciliary epithelial cells of the eye; corneal endothelial cells; ciliated cells having propulsive function; ameloblasts; planum semilunatum cells of the vestibular apparatus of the ear; interdental cells of the organ of Corti; fibroblasts; pericytes of blood capillaries; nucleus pulposus cells of the intervertebral disc; cementoblasts; cementocytes; odontoblasts; odontocytes; chondrocytes; osteoblasts; osteocytes; osteoprogenitor cells; hyalocytes of the vitreous body of the eye; stellate cells of the perilymphatic space of the ear; skeletal muscle cells; heart muscle cells; smooth muscle cells; myoepithelial cells; red blood cells; platelets; megakaryocytes; monocytes; connective tissue macrophages; Langerhans cells; osteoclasts; dendritic cells; microglial cells; neutrophils; eosinophils; basophils; mast cells; plasma cells; helper T cells; suppressor T cells; killer T cells; killer cells; rod cells; cone cells; inner hair cells of the organ of Corti; outer hair cells of the organ of Corti; type I hair; cells of the vestibular apparatus of the ear; type II cells of the vestibular apparatus of the ear; type II taste bud cells; olfactory neurons; basal cells of olfactory epithelium; type I carotid body cells; type II carotid body cells; Merkel cells; primary sensory neurons specialised for touch; primary sensory neurons specialised for temperature; primary neurons specialised for pain; proprioceptive primary sensory neurons; cholinergic neurons of the autonomic nervous system; adrenergic neurons of the autonomic nervous system; peptidergic neurons of the autonomic nervous system; inner pillar cells of the organ of Corti; outer pillar cells of the organ of Corti; inner phalangeal cells of the organ of Corti; outer phalangeal cells of the organ of Corti; border cells; Hensen cells: supporting cells of the vestibular apparatus; supporting cells of the taste bud; supporting cells of the olfactory epithelium; Schwann cells; satellite cells; enteric glial cells; neurons of the central nervous system; astrocytes of the central nervous system; oligodendrocytes of the central nervous system; anterior lens epithelial cells; lens fiber cells; melanocytes; retinal pigmented epithelial cells; iris pigment epithelial cells; oogonium; oocytes; spermatocytes; spermatogonium; ovarian follicle cells; Sertoli cells; and thymus epithelial cells; hepatocarcinoma; any cell line derived therefrom; or any combination thereof.

In certain embodiments, the encapsulated cells are mammalian cells. For example, the encapsulated cells may be human, bovine, equine, ovine, murine, from a primate, or from a rodent.

In preferred embodiments, the cells are stem cells. The stem cells may be pluripotent stem cells. The stem cells may be human stem cells. The stem cells may be human embryonic stem cells. The stem cells may be induced pluripotent stem cells.

The number of cells encapsulated in a given fiber will generally depend on factors such as the length and diameter of the fiber, the size and morphology of the cells encapsulated, the desired cell density, and so on. Preferably, the fibers comprise a high to density of cells, although the density of cells will depend on the particular application.

In certain embodiments, the fiber comprises encapsulated cells at number of between about 50 million and 500 million cells/ml of total solution used to draw the fiber. In other embodiments, the fiber comprises a cell density of between about 50 million and about 250 million cells/ml of total solution used to draw the fiber, or between about 100 million and about 200 million cells/ml, between about 100 million and about 150 million cells/ml, between about 10 million and about 100 million cells/ml, or between about 20 million and about 50 million cells/ml of total solution used to draw the fiber.

In addition to encapsulated cells, fiber-assembled tissue constructs of the invention may comprise other additional components. The additional components may or may not be encapsulated. As noted above encapsulated components will generally be unable to move out of the fiber while the integrity of the fiber is maintained, although they may be capable of migrating within the fiber. It follows that components which are not encapsulated in the fiber may move in or out of the fiber, as well as within the fiber.

In certain embodiments, the fibers may comprise a biologic, or, a mixture of different biologics. The biologics may be encapsulated. Encapsulated biologics may be conjugated to the fiber material and thus may generally only diffuse out of the fiber once the fiber degrades. Alternatively, the biologics may not be encapsulated and thus be capable of moving out of the fiber.

Non-limiting examples of suitable biologics include proteins (e.g. extracellular matrix proteins such as collagen, elastin, pikachurin; cytoskeletal proteins such as actin, keratin, myosin, tubulin, spectrin; plasma proteins such as serum albumin; cell adhesion proteins such as cadherin, integrin, selectin, NCAM; and enzymes), hormones and other growth factors (e.g. insulin, insulin-like growth factor, epidermal growth factor, oxytocin); neurotransmitters (e.g. serotonin, dopamine, epinephrine, norepinephrine, acetylcholine); angiogenic factors (e.g. angiopoietins, fibroblast growth factor, vascular endothelial growth factor, matrix metalloproteinase enzymes); amino acids; galactose ligands; nucleic acids (e.g. DNA, RNA); and drugs (e.g. antibiotics).

The biologics may be obtained from any source (e.g. humans, other animals, microorganisms). For example, they may be produced by recombinant means or may be extracted and purified in natural from directly from a living source. It is also contemplated that different encapsulated cell types within fibers of the invention may provide a source of the additional components.

### Secondary and Tertiary Structures

In certain embodiments of the present invention, multiple fibers may be arranged to form secondary or tertiary structures. For example, two, three, four, five, six, seven, eight, nine, ten or more than ten fibers may be arranged in any manner to form a secondary structure (e.g. by twisting multiple fibers together along a central longitudinal or vertical axis; by wrapping one or more fibers around one or more central fibers). Secondary structures formed from multiple indicidual fibers may be combined to form tertiary structures.

### Degradable Fibers

Fibers according to the present invention may be degradable to allow for the release of encapsulated components. In this context, the term “degradable” will be understood to mean that the fiber has the potential to be modified in a manner that facilitates the release of encapsulated components. Reference herein to a component (e.g. a cell) that is “releasably encapsulated” will be understood to mean a component encapsulated in a fiber that can be modified in a manner facilitating release of the encapsulated component.

For example, polymerised material within the fiber may be degraded by the application of a suitable agent capable of cleaving covalent bonds existing between monomer units and/or between cross-linked polymer chains. Preferably, the agent is biocompatible in the sense that it has minimal detrimental effect on the integrity or function of encapsulated components of the fiber. In the case where encapsulated components are cells (e.g. stem cells), a biocompatible agent used to degrade the fiber preferably has little or no detrimental impact on the viability of the cells, their capacity to propagate or their capacity to differentiate. This may be determined using standard techniques known to the skilled addressee, including those described in the Examples of the present specification. For example, the capacity of pluripotent stem cells to differentiate may be determined by assessing their ability to form derivatives of the three germ layers (see also Example 1 of the present specification).

In certain embodiments, a fiber of the present invention may be degradable by one or more of biological degradation, chemical degradation, photodegradation or thermal degradation.

Preferably, a fiber of the present invention is susceptible to biological degradation. For example, one or more enzymes may be applied to the fiber for the purpose of cleaving covalent bonds existing between monomer units and/or between cross-linked polymer chains. Any enzyme capable of performing this function could potentially be used, depending on the specific nature of the polymers in the fiber.

In certain embodiments, the fiber comprises chitin and/or alginate susceptible to degradation with a biological agent. The biological agent may be an enzyme. The enzyme may be a chitinase or an alginate lyase (also known as alginate depolymerase and alginate).

Additionally or alternatively, a fiber of the present invention may be susceptible to chemical degradation such as, for example, oxidation, solvolysis, or hydrolysis. Chemical degradation may be achieved by the application of a suitable agent such as, for example, an acid, alkali and/or salt.

Fibers of the present invention may be degraded using combinations of different agents. The different agents may be applied simultaneously or successively.

### Preparation of Fibers

Fibers according to the present invention may be prepared using methods known in the art.

In preferred embodiments, fibers of the invention may be fabricated (i.e. formed) by drawing up from an interface formed between two oppositely charged polyelectrolyte solutions. Factors such as the appropriate polyelectrolyte concentration in a given solution, the relative volumes of polycationic and polyanionic solutions, the molecular weight of the polycationic/polyanionic materials selected, the pH and temperature of each solution, the viscosity of each solution, the means of drawing the fiber, and the rate at which the fiber is drawn, can be readily determined by the skilled addressee without exercising inventive effort (see, for example, experimental data provided herein and references [1] and [2] referred to in Example 1 of the present specification).

By way of non-limiting example, in certain embodiments of the invention a fiber may be formed by dispensing a droplet from a polycationic solution adjacent to a droplet from a polyanionic solution onto a suitable surface. The droplets may be positioned closely together but slightly apart. Depending on the intended application of the fiber, either or both of the polyanionic and polycationic solutions may comprise components such as, for example, cells and/or biologics.

Opposing surfaces of each adjacent solution may each be contacted with the tip(s) of a pointed instrument (e.g. a pipette tip, needle, pair of forceps, etc.) and the opposing surfaces of each droplet brought together to form an interface.

The tip(s) may be coated with an adhesive to allow adherence to a fiber drawn from the polyelectrolyte solutions. In general, the adhesive may be any material capable of assisting contact (directly or indirectly) between the tip and the polyelectrolyte solutions. Any suitable adhesive may be used, including organic and inorganic materials. Non-limiting examples of organic adhesives that may be used include fibrin glue, polyvinylpyrolidone, polyvinylpyrolidone/vinyl acetate copolymers, cyanoacrylate gel, platelated gel, chitosan or gelatin-resorcin-formaldehyde (GRFG). Additional non-limiting examples include organic polymeric compositions represented by the group of alkyd resins, polyvinyl acetaldehydes, polyvinyl alcohols, polyvinyl acetates, poly(ethylene oxide), polyacrylates, ketone resins, polyvinylpyrolidone, polyvinylpyrolidone/vinyl acetate copolymer, polyethylene glycols of 200 to 1000 molecular weight and polyoxyethylene/polyoxopropylene block copolymers (Polyox), silicone resins and silicone based pressure sensitive adhesives. Pressure sensitive adhesives are well known in the art and commercially available (e.g. those available from Dow Corning Company under the trade designation BIO-PSA).

A nascent fiber may commence forming from the interface by continued upward drawing. The fiber may be drawn upwards at an appropriate rate (e.g. between about 1 mm and about 10 mm/second, between about 5 mm and about 10 mm/second about 5 mm/second, about 1 mm/second, about 0.5 mm/second or about 0.3 mm/second). The nascent fiber may optionally be drawn through a suitable solution (e.g. calcium chloride. hydrolysed tetraethoxysilane) to reduce secondary complexion of the components of the fiber. Application of such a solution to the fiber may serve to crosslink polyelectrolytes within the fiber, prevent swelling of the fiber, and/or decrease interactions between polyelectrolytes from different fibers. Accordingly, the application of such a solution may serve to increase the mechanical strength of individual fibers.

The fiber may be optionally drawn through a second solution (e.g. PBS) to wash the first solution from the fiber.

Continued upward drawing of the fiber may be used to lengthen the nascent fiber. Preferably, drawing of fibers is conducted in a humid atmosphere to protect cells and other constituents within the fibers from drying.

The nascent fiber may be spooled onto an appropriate fiber holder (e.g. a disk, rod, sheet, cube etc.). The holder may comprise dimensions of about 0.7 cm×about 1.0 cm. The fiber may be a continuous fiber or a discontinuous fiber. Preferably, the fiber is a continuous fiber.

In the case where a fiber with encapsulated cells is to be formed, it may be preferable to perform the process at room temperature and near-neutral pH aqueous solutions to preserve the integrity and viability of the cells.

### Culture of Fibers

Fibers of the present invention may be cultured to facilitate propagation and/or differentiation of cells encapsulated within the fibers. Any suitable media for cell culture may be used for this purpose.

In certain embodiments, a fiber of the invention may be provided as a component of a buoyant fiber assembly capable of suspension in culture media. Preferably, the assembly is negatively or neutrally buoyant in the media. Most preferably, the assembly is neutrally buoyant in the media which may maximise the capacity for uniform exposure of cells encapsulated in the fibers to soluble factors present in the media, consequently facilitating efficient propagation and/or differentiation of encapsulated cells.

For example, in some embodiments fibers of the present invention are spooled onto an appropriate fiber holder (e.g a planar PVDF membrane). Preferably, the mass of the fiber holder with spooled fiber equals the mass that it displaces in the surrounding culture medium (i.e. it is neutrally buoyant in the culture medium).

In some embodiments, a fiber assembly may comprise a fiber comprising encapsulated stem cells (e.g. encapsulated pluripotent human embryonic stem cells). The assembly may be cultured in media comprising soluble factors capable of supporting or enhancing propagation of the encapsulated stem cells, without causing substantial differentiation of the stem cells (i.e. the stem cells increase in number but do not lose pluripotency). Alternatively, the assembly may be cultured in media comprising soluble factors capable of supporting or enhancing differentiation of the encapsulated stem cells into one or more specific lineage(s). Alternatively, the assembly may be cultured in media comprising soluble factors capable of supporting or enhancing the propagation of encapsulated stem cells and their differentiation into one or more specific lineage(s). Suitable culture media, soluble factors and culture conditions to achieve such outcomes are well known to those of skill in the field (see also Example 1 of the present specification). Moreover, tests and assays capable of measuring cell propagation and/or differentiation are also well known in the art. For example, cells may be enumerated under microscopy or by flow cytometry. The degree of differentiation of the cells may be assessed, for example, by profiling the expression of cell markers and/or in the case of stem cells assessing the ability to form derivatives of the three germ layers (see also Example 1 of the present specification).

In certain embodiments, fibers of the present invention may be used for the direct differentiation of encapsulated cells into tissue-like structures.

### Cryopreservation of Fibers

Fibers of the present invention may be cryopreserved. The fibers may comprise encapsulated cells. The fibers may be components of a fiber assembly suitable for cell culture.

Suitable methods for cryopreservation are well known in the field, and are also described in Example 1 of the present specification.

Cryopreserved fibers, which may comprise encapsulated cells, and which may be components of a fiber assembly suitable for cell culture, can be thawed using standard techniques and re-introduced to culture media for further re-culturing and/or the release of encapsulated cells. The skilled addressee will be well acquainted with suitable techniques for thawing cryopreserved biological material, including the methodology disclosed in Example 1 of the present specification.

### Exemplary Embodiments

By way of non-limiting example only, a fiber according to the present invention may be formed according to the following.

A polycationic alginate solution may be prepared at a concentration of between about 0.1% to about 5%, preferably between about 0.5% to about 3%, and more preferably between about 1.5% to about 2.5% (e.g. about 2%). The alginate solution may be prepared utilising, for example, a suitable salt (e.g. sodium alginate, calcium alginate and the like). The viscosity of the polycationic solution may be between about 20 cps and about 200 cps.

Water soluble chitin may be used to prepare a polyanionic solution at a concentration of between about 0.1% to about 5%, preferably between about 0.5% to about 3%, and more preferably between about 1.5% to about 2.5% (e.g. about 2%). The chitin may be derived from suitable material such as, for example, crab shell. The chitin may be deacetylated, or partially deacetylayted (e.g. about 10%, 20%, 30%, 40%. 50%, 60%, 70% or 80% deacetylated; or between about 10% and about 50% deacetylated; between about 30% and about 60% deacetylated; or between about 45% and about 55% deacetylated). The viscosity of the polyanionic solution may be between about 200 cps and about 300 cps. The viscosity of the polyanionic solution may be about 250 cps.

At least one of the polycationic or the polyanionic solution may comprise suspended cells. The cells may be stem cells, preferably embryonic stem cells and still more preferably human embryonic stem cells. The stem cells may be pluripotent stem cells. The cells may be suspended at between about 2×10⁷ and about 5×10⁷ cells/ml of solution. The cells may be suspended in the polycationic alginate solution and not the polycationic chitin solution.

A nascent fiber comprising encapsulated cells may be formed by drawing up an interface formed between opposing surfaces of the polyanionic and polycationic solutions. The nascent fiber may optionally be passed through a first solution of CaCl₂ (e.g. 50 mM) and a second solution of PBS, consecutively, attached onto a sterilized fiber holder, and spooled onto the holder. The fiber may be cultured in an appropriate medium suitable for the propagation and/or differentiation of encapsulated cells. The fiber holder is submersed in the culture medium and preferably adopts a neutrally buoyant position in the medium to allow the encapsulated cells uniform exposure to soluble factors in the medium that facilitate efficient propagation and/or differentiation of the encapsulated cells.

After culturing for a suitable time period, encapsulated cells may be released by treating the construct with an appropriate chemical or enzyme (e.g. chitinase and/or alginate lyase).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLE

The invention will now be described with reference to a specific example, which should not be construed as in any way limiting.

### Example 1

### Engineered Three-Dimensional Fibrous Encapsulation Culture System which Supports Large-Scale Efficient Expansion, Isolation and Controlled Differentiation of hESCs

### Materials and Methods

2.1. hESC Culture

ES cell lines, the green fluorescent protein(GFP)-expressing hESC cells BG01 V/hOG hESCs (Invitrogen), and HUES9 were cultured on a feeder layer of mitomycin c-treated mouse embryonic fibroblasts (MEFs) (ATCC, USA) in ES culture medium (FCM) which consisted of 80% KNOCKOUT Dulbecco's Modified Eagles Medium (KO-DMEM, Gibco), 20% KNOCKOUT serum replacement (KSR) (Invitrogen), 1% non essential amino acid solution (Invitrogen), 1 mM Glutamax (Invitrogen), 1% penicillin/streptomycin (100 U/ml/0.1 mg/ml), 0.1 mM (3-mercaptoethanol and 4 ng/ml basic fibroblast growth factor (bFGF). Daily medium changes began 48 hours post seeding. hESCs were sub-cultured every 6 days with trypsin-EDTA (Sigma).

2.2. hESC Encapsulation Using Chitin-Alginate Fibrous Substrates

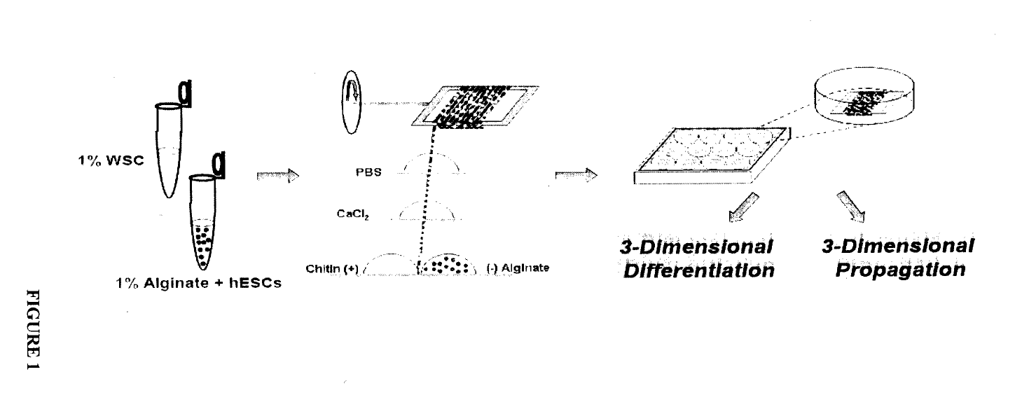

Previous fiber encapsulation methods were modified for hESC encapsulation (FIG. 1) [1]. Typically, the microfibers for cell encapsulation were formed by the interfacial electrostatic interaction of alginate and chitin solution. 2% alginate (Sigma-Aldrich) and 1% water-soluble chitin (WSC) in PBS [3] were used in this study. In certain embodiments, and as exemplified herein, the WSC is chitin with a degree of deacetylation of approximately 50%. Based on the experimental scale, the fiber holders were prepared by cutting PVDF membrane to fit into tissue culture plates (1.5 cm×1 cm rectangle with 0.25 cm edge for 12-well culture plate). The fiber holders were sterilized with 70% ethanol and air-dried in tissue culture hood.

A confluent monolayer of adherent ES cells was harvested following trypsin incubation. The cell suspension was filtered with 40 μm cell strainers and re-suspended in culture medium. For cell encapsulation, 2˜5×10⁷ cells/ml of hESCs were mixed with 2% alginate PBS solution to obtain a suspension of cells in 1% alginate. The hESCs could alternatively be suspended in the WSC solution, or in both the alginate and WSC solutions. Cell-fiber constructs were fabricated by drawing up the interface between the two oppositely charged polyelectrolyte solutions using a pair of forceps, passed as a continuous fiber through CaCl₂ (50 mM) and PBS washing baths consecutively, and then attached to a sterilized fiber holder. The cell-fiber constructs were cultured in MEF conditioned medium (CM) [4] with daily change of cell culture medium. Cell viability and morphology was observed under florescence microscopy.

2.3. hESC Recovery, Subculture and Enzyme Cytotoxicity

Chitinase (Sigma) and alginate lyase (Sigma) were used to decapsulate the cell-fiber constructs. The enzyme cytotoxicity was evaluated by the MTT assay kit [3-(3,4-dimethylthiazol-2-yl)-2,5-diphenyltetrazoliium bromide, Sigma]. The enzymes were dissolved in KO-DMEM and sterile-filtered separately. The solutions were diluted in hESC culture medium to concentrations between 0 and 0.5 U/mL (chitinase) or 145 U/mL (alginate lyase). hESCs were seeded on matrigel-coated 96-multiwell tissue culture dishes at a density of ˜40,000 cells/cm². Following 48 h of culture at 37° C. and 5% CO₂, the medium was replaced by fresh medium containing chitinase, alginate lyase or their mixture at the indicated concentrations. Matrigel-coated wells with fresh medium without substrate served as controls. Following 0.5 h in culture, cells were cultured in the medium containing 1 mg/mL MTT solution. After incubation at 37° C. for 4h, the blue formazan crystals were dissolved using MTT buffer. The absorbance of the solution was measured using a microplate reader (Spectra Count™) at 590 nm.

To release the encapsulated hESCs, cell-fiber constructs were incubated with cell culture medium containing various concentrations of chitinase and alginate lyase for 5 mins at 37° C., followed by centrifugation for 3 mins at 200g to remove the fiber debris. Cell viability and recovery efficiency were determined under florescence microscopy.

For cell passage, the recovered cell clusters were dissociated into single or small cell aggregates by incubating in 1 mg/ml dispase (Sigma) and encapsulated using the cell culture protocol described above.

2.4. hESC Viability and Proliferation in Chitin-Alginate Microfibers

To evaluate hESC propagation in the fibers, encapsulated cell clusters were recovered using chitinase-alginate lyase decapsulation method. Approximately 100,000 hESCs were encapsulated in chitin-alginate fibers and cultured for 14 days as described in section 2.2 above. At fixed times, the encapsulated cells were recovered using chitinase-alginate lyase working solution as described above. Recovered cell clusters were dispersed into single cells using Accquat (Sigma) and counted under microscopy. The same amount of hESCs cultured on Matrigel coating was used for comparison. Trypan blue exclusion method was applied to determine cell viability.

### 2.5. Flow Cytometry

For fluorescence-activated cell sorting (FACS) analysis, 1×10⁵ BG01 V/hOG hESCs were encapsulated into chitin-alginate fibers and cultured for 2 weeks. Cell clusters were released from fibers using the chitinase-alginate lyase decapsulation method and dispersed into single cells in FACS buffer using Accquat. hESCs cultured on MEF feeder layer and Matrigel-coated plate for 6 days were used for comparison. Cells were analyzed with a BD fluorescence-activated cell sorting (FACS) flow cytometer.

### 2.6. Immunostaining Assay

Undifferentiated or induced differentiated hESCs were released from fibers, fixed and immunostained by standard indirect immunochemistry. Cell membranes were permeabilized with 0.1% Triton-X100 for 30 min at room temperature. The cells were then blocked with 3% BSA containing PBS buffer for 1 h at room temperature. Incubation was carried out overnight at 4° C. with rabbit anti-Nanog (Santa Cruz), mouse anti-βIII tubulin (Promega), and rabbit anti-neurofilament L (Millipore). After three washes with PBS for 5 mins, secondary antibodies containing DAPI as nuclear DNA staining were applied for 1 h at room temperature. Donkey anti-mouse, -rabbit secondary antibodies conjugated with FITC or TR were used. The samples were observed under a Zeiss LSM510 laser scanning microscope and photographed and processed with LSM Image Browser software.

### 2.7. RNA Extraction and RT-PCRs

Total RNA was isolated using Trizol reagent (Invitrogen) according to manufacturer's instruction. Before reverse transcription. RNA samples were digested with DNase I (Invitrogen) to remove contaminating genomic DNA. cDNA synthesis was performed using SuperScript III Reverse Transcriptase and Oligo (dT) primers (Invitrogen). Human specific PCR primers for Oct3/4. Nanog and GADPH were purchased from Taqman with the sequence listed in Table 1. Cycling conditions were: initial denaturation at 95° C. for 2 min, followed by 35 cycles of denaturation (30 s at 95° C.), annealing (60 s at 55° C. or 59° C. depending on the primer), elongation (90 s at 72° C.). followed by a final 10 min elongation at 72° C. ES cells cultured on MEF feeder layers were used as the control.

TABLE 1
the primers of selected genes for PCR

| Primer | Sequence | |
|---|---|---|
| NF68 - Forward | 5′- AGAAAGTGCACGAAGAGGAGATCG -3′ | SEQ ID NO: 1 |
| NF68 - Reverse | 5′- ATCTTCACGTTGAGGAGGTCTTGG -3′ | SEQ ID NO: 2 |
| CNPase - Forward | 5′- TGGAAACTTCCAAGACCTGACTC -3′ | SEQ ID NO: 3 |
| CNPase - Reverse | 5′- CAGAGATAAAACCCTGCCTCCTGA -3′ | SEQ ID NO: 4 |
| Tau (MAPT) - Forward | 5′- AGACACTGTTCCCAAAGCCTTGAC -3′ | SEQ ID NO: 5 |
| Tau (MAPT) - Reverse | 5′- ACCCCACATTTCCTTCTCCTTCTC -3′ | SEQ ID NO: 6 |
| GAPDH - Forward | 5′- AGCCACATCGCTCAGACACC -3′ | SEQ ID NO: 7 |
| GAPDH - Reverse | 5′- GTACTCAGCGGCCAGCATCG -3′ | SEQ ID NO: 8 |
| Oct4 - Forward | 5′- CTTGCTGCAGAAGTGGGTGGAGGAA -3′ | SEQ ID NO: 9 |
| Oct4 - Reverse | 5′- CTGCAGTGTGGGTTTCGGGCA -3′ | SEQ ID NO: 10 |

2.8. Assessment of Pluripotency of Recovered hESC In Vivo

To assess whether the encapsulated hESCs maintain pluripotency, the released cell clusters from 14-day post-encapsulated fibers were implanted subcutaneously in SCID mice (˜5×10⁶ cells per mouse). All experimental procedures were approved, following the guidelines of the National Advisory Committee on Laboratory Animal Research (NACLAR). After 8 weeks, teratomas were removed, and processed carefully for RNA extraction and histological analysis.

Gene expression analysis of teratomas was carried out RT²Profiler™ PCR array (SuperArray Bioscience Corporation). 2 ug total RNA from the tissues were converted into cDNAs using RT² first strand kit. Briefly, DNase treated RNAs reverse transcribed at 42° C. for 15 min. The enzyme was heat-inactivated at 95° C. for 5 min. The cDNAs were diluted and used in the real-time PCR. Real-time PCR was performed in a iQ™ 5 Multicolor Real-time PCR Detection System (Bio-Rad) using RT² SYBR Green master mix. Amplification was performed in a total volume of 25 μl. After a cycle of 95° C. for 10 min, the reactions were cycled 40 times under the following parameters: 95° C. for 15 s. 60° C. for 1 min. At the end of the PCR, the melting curve program was ran at 95° C. for 1 minute, 55° C. for 2 minutes followed by a gradual temperature increment of 0.5° C. per minute from 55° C. to 95° C. A non-template control (NTC) was run with every assay, and gene expression for teratomas from two SCID mice were performed separately. Data analysis was performed using online SABiosciences and Cluster [5] software, viewed by TreeView software [6].

2.9. In Vitro Direct Induction of Neuronal Differentiation of Encapsulated hESCs

Seven-day propagated undifferentiated hESC clusters in chitin-alginate fibers were treated with neuron stimulation medium (NSM: KNOCKOUT DMEM/F12 supplied with N2, B27, Glutamax, NEAA, 100 ng/ml recombinant mouse Noggin (R & D Systems) and 2 μM retonic acid. Retinoic acid and Noggin can promote neural differentiation. After 7 days of induction, the cell-fiber constructs were cultured in neural progenitor differentiation medium (NPM) (Chem con) for another 10 days. Differentiated cell clusters were released from fiber using the enzymatic method described in section 2.3 above, and proceeded to subculture, immunostaining or RNA extraction.

For subculture, the differentiated cell clusters were incubated with dispase at 37° C. for 10 mins to disperse into single or small clusters. The cells were pelleted and plated on ornithine/laminin substrates in NPM. Morphological analyses and immunostaining with markers for mature neural cells were performed.

Gene expression analysis of differentiated hESCs was conducted following the method described in section 2.8. above. Primer sequences are listed in Table 1 above.

2.10. hESC Cryopreservation in Chitin-Alginate Fibers

### Freezing in Chitin-Alginate Fibers

Six-day-culture hESC-fiber constructs were carefully transferred into cryovials containing 1 ml mFreSR® (STEMCELL technologies) or the prepared freezing medium (10% DMSO, 40% KNOCKOUT serum replacement, 50% CM) via a sterile forcep. The cryovials were kept at room temperature for 10 mins, transferred to a Nalgene Cryo 1° C. Freezing container (Fisher Scientific) and frozen in a 80° C. freezer. The frozen hESCs were transferred into liquid nitrogen for long-term storage the next day.

### Thawing

Cryovials were quickly thawed in a 37° C. water bath. The thawed hESCs-fiber constructs were carefully transferred to ES fresh culture medium for further expansion or differentiation. For subculture, hESC aggregates were released from fibers using chitinase-alginate lyase method as described in 2.3, dispersed into small aggregates using trypsin, and plated onto a MEF feeder layer on a culture plate or Matrigel-coated culture plate.

### 2.11. Statistical Analysis

Data expressed as means±S.D. Statistical significance between two groups was determined by the unpaired Student's t-test. Results for more than two experimental groups were evaluated by one-way ANOVA to specify differences between groups. P<0.05 was considered significantly different.

### Results and Discussion

3.1 Microfiber Encapsulation and Morphological Analysis of hESCs

These experiment aimed to develop a scalable 3D culture system to generate sufficient quantities of hESCs and derivatives for possible clinical application. To achieve this target, fiber engineering was integrated with a cell encapsulation approach, as shown in FIG. 1. The approach affords the following advantages: 1) employing natural polymers and physiological encapsulation process. 2) engineering 3D fiber assembly planar formats to provide uniform exposure to soluble medium components. 3) encapsulating a high density of cells into 3D fibrous substrate which can be easily incorporated with ECM for cell growth. 4) Developing user-controllable isolation method while preserving 3D multicellular tissue structure.

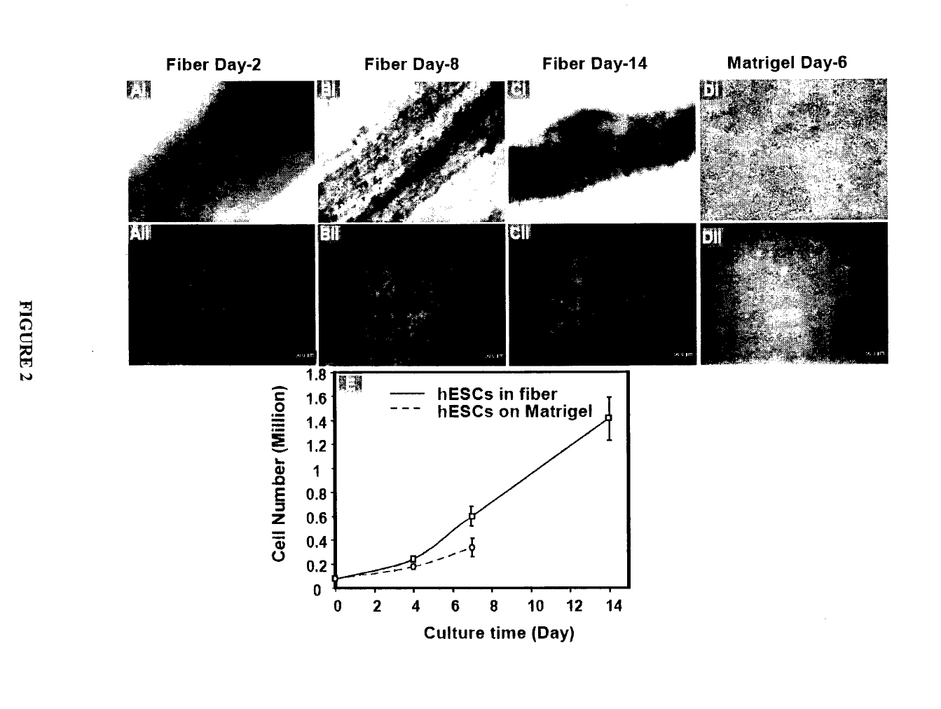

The HESC line H9 and BG01 V/hOG hESCs were chosen to test the approach. BG01 V/hOG hESCs are engineered to enable monitoring of pluripotency. These cells express Emerald Green Florescent Proteins (emGFP) when pluripotent and lose GFP expression upon differentiation. Passage 6 hESCs were harvested and encapsulated in 1% chitin-alginate fibers. FIG. 2 shows the typical appearance of hESCs after encapsulation in comparison to those cultured on Matrigel layer. After encapsulation, human ESCs were uniformly distributed in chitin-alginate microfibers and formed cell colonies/clusters which proliferated with time in culture (FIG. 2A-C). In contrast, hESCs cultured on the Matrigel coated layer assumed a monolayer morphology (FIG. 2D). The strong green fluorescence from encapsulated hESCs suggested undifferentiated cell status. hESCs encapsulated in chitin-alginate fiber exhibited higher proliferation rate than hESCs cultured on matrigel-coated layers (FIG. 2E).

3.2 hESCs Recovery Using Chitinase-Alginate Lyase Decapsulation Method and Subculture

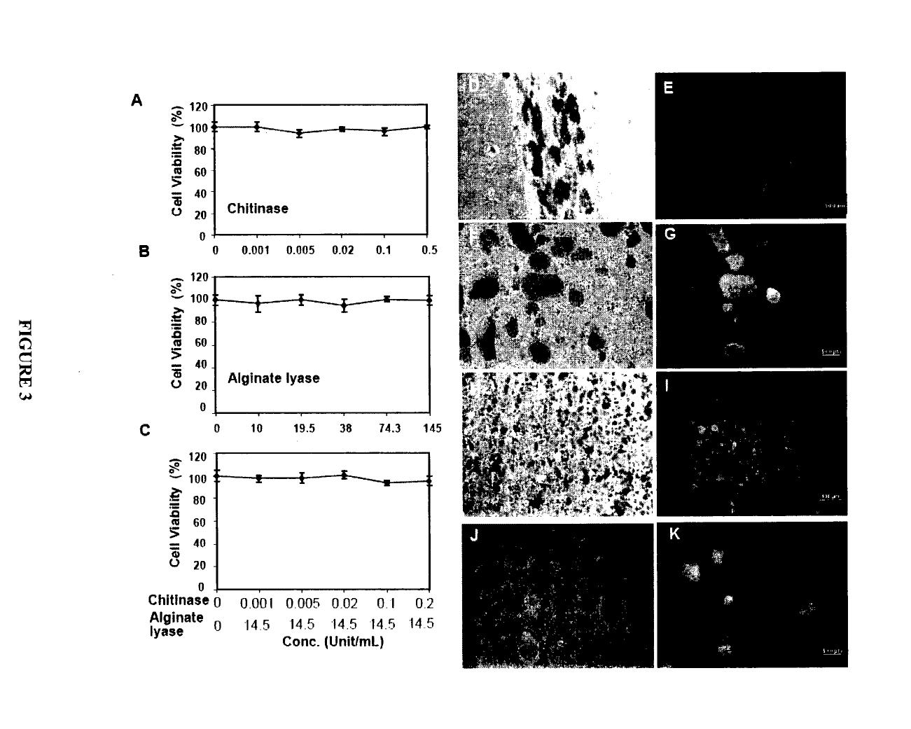

One challenge of hESC culture in 3D culture scaffolds is to expand them to high cell densities and following that, to harvest them easily for further animal studies and possible regenerative therapies. In the present study, the encapsulated hESCs could be released by treatment with a mixture of chitinase-alginate lyase. Since hESCs are particularly vulnerable to harmful culture conditions, the possible toxicity of the enzymes during the cell recovery process was first assessed. hESCs seeded on Matrigel-coated layer were incubated with culture medium containing a range of concentrations of enzyme and cell viability was measured using the MTT assay. As shown in FIG. 3A-C, hESCs incubated with culture medium containing 0-0.5 Units/mL chitinase and 0-145 Units/ml alginate lyase or combinations thereof preserved their normal morphology with negligible loss of viability, suggesting that the recovery method by using chitinase-alginate lyase was safe and practicable.

Next the optimum concentration of enzyme for fiber degradation and hESC recovery was tested. After incubating hESCs with medium containing various concentrations of enzyme mixture at 37° C. for 5 mins, it was found that a mixture of 0.1 Unit/ml Chitinase and 14.5 Units/mL alginate lyase resulted in complete degradation of fibers (FIG. 3D-I). The hESCs released from fibers exhibited strong green fluorescence, suggesting the full preservation of cell viability and undifferentiated phenotype. These released hESC clusters could readily adhere to MEF layers and proliferated at similar rates as seen in standard monolayer cultures (FIGS. 3J and K).

3.3. Released hESCs Maintain Self-Renewal Potency

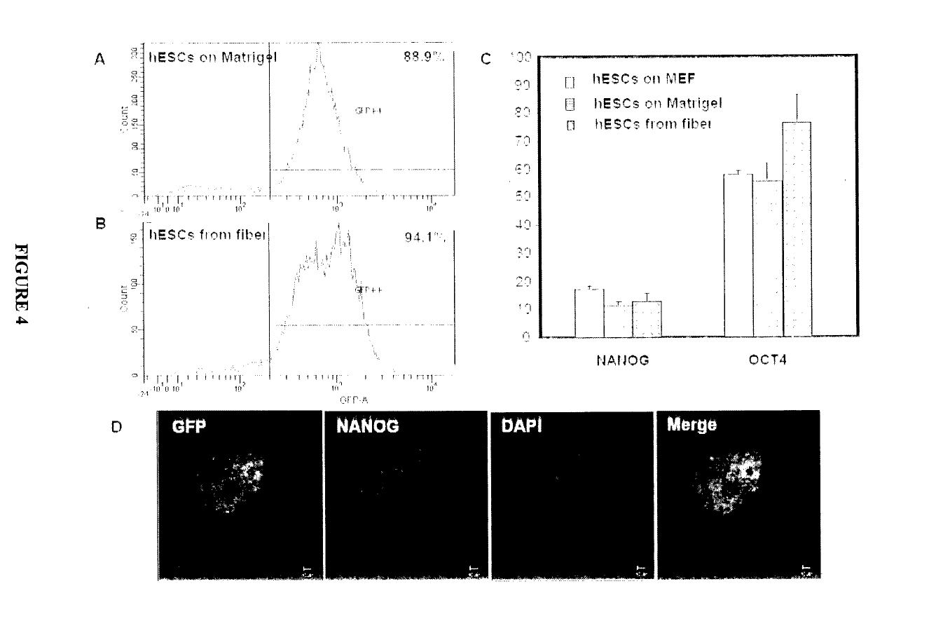

Since the BG01 V/hOG hESCs contained GFP which can be a marker for self-renewal status, the released hESCs from fibers were characterized by flow cytometry. FIGS. 4A & B show the quantification results of hESCs cultured in microfibers compared with those cultured on Matrigel layers. The level of GFP-positive cells released from chitin-alginate fibers was comparable to that cultured on Matrigel layers (94.1% vs 88.9%), suggesting that the fibrous scaffold can support hESC self-renewal and propagation.

Gene expression and immunostaining studies were performed to further confirm HESC pluripotency. RNA samples were taken from hESCs cultured in fibers, on Matrigel and MEF layers, and the relative expression of stem marker genes Oct4 and Nanog was studied. Real-time PCR analysis revealed that the released hESC clusters expressed the stem cell pluripotent marker genes Oct 4 and Nanog at a level comparable to those expressed by hESCs grown on Matrigel or MEF layers (FIG. 4C). This result is consistent with the high expression of Nanog at the protein level in the released hESC clusters, revealed by immunostaining (FIG. 4D). Collectively, these results suggest that the fiber culture system developed supports hESC growth while maintaining self-renewal potency.

3.4 Released hESC Clusters Exhibit Pluripotency In Vivo

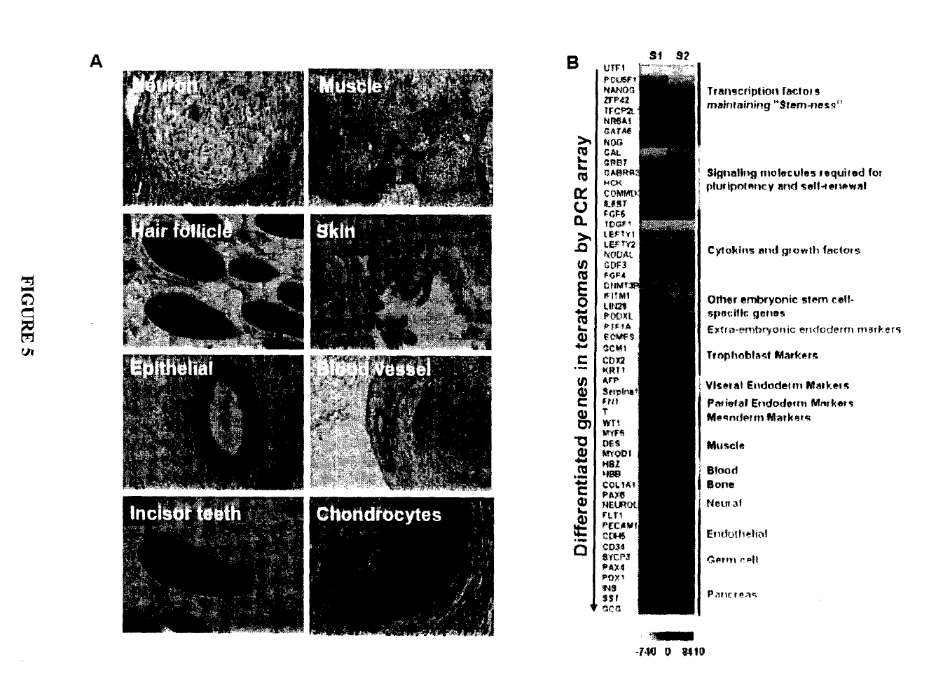

To assess the potential of hESC clusters released from chitin-alginate fibers to form teratoma and differentiate to ectoderm, mesoderm and endoderm, 2-week encapsulated hESCs were released and implanted subcutaneously in NOD/SCID mice. The mice were sacrificed 2 months following implantation and the formed teratoma were collected and characterized. Histological images revealed the formation of differentiated cells and tissues derived from the three germ layers including neuron, skin, muscle, chondrocyte, epithelial and blood vessel (FIG. 5A).

To get a better idea of how hESCs differentiated into multilineage tissue in vivo, quantitative PCR was used to measure the expression of key genes involved in the maintenance of pluripotency and the self-renewal status of embryonic stem cells using an RT² Profiler™ PCR array. The gene expression profile of the teratoma was compared after 2 month implantation with that of the control, self-renewing hESCs. Among the expression genes detected, 50 genes showed fold change of more than 4 times in either direction (p<0.05). Marked down-regulation of embryonic stem cell-specific genes and parallel up-regulation of selected embryonic stem cell differentiation/lineage markers were observed (FIG. 5B)

3.5 Direct Differentiation of hESCs Encapsulated in Fibrous Scaffolds

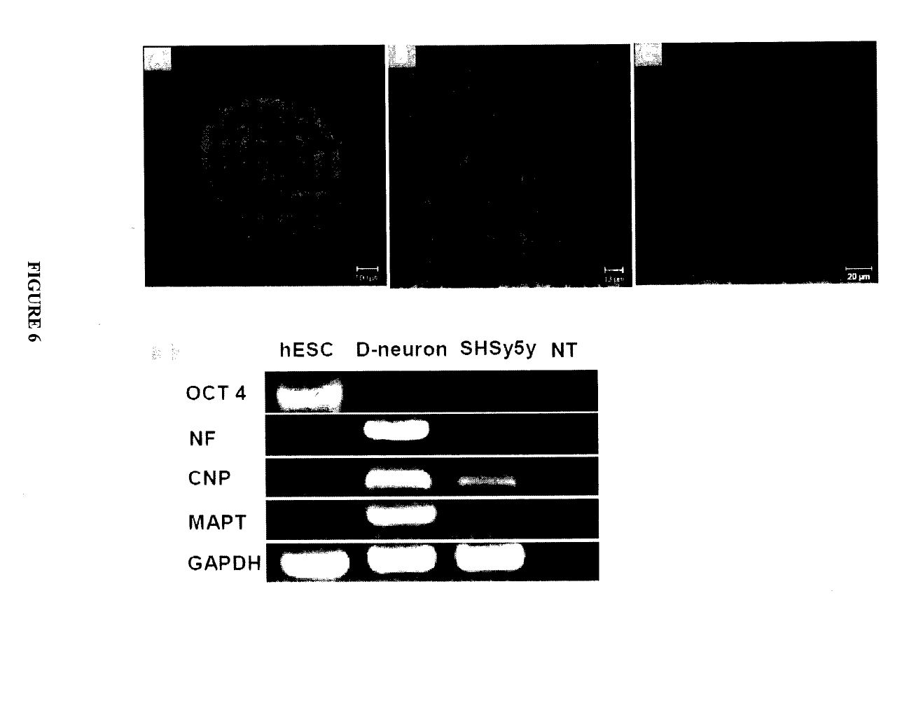

The desired approach for controlling hESC differentiation is to mimic the 3D embryogenesis environment, where hESC expansion and differentiation happens within the same 3D system. It was tested whether the hESC clusters expanded in chitin-alginate fibers could be directed to differentiate into a specific lineage in the form of tissue-like multicellular structures. For this purpose, the encapsulated hESCs were cultured for one week in hESC culture medium, then incubated with culture medium containing noggin and RA for 10 days. After another 10-day treatment with neuron differentiation medium, the clusters were released and seeded on a laminin substrate in the same neuron differentiation medium. After 12-h plating, numerous neurites were observed to extend out from the cluster cells. By 2-day post seeding, multilayer neuronal networks had been formed through cell processes. RT-PCR study revealed that the released clusters expressed neuron specific genes including TF, CNP, and MAPT, but not the embryonic stem gene, OCT4 (FIG. 6D). Immunostaining analyses indicated that the differentiated cell clusters were positive for neuron specific markers bIII-tubulin and neurofilament (FIG. 6A-C). Collectively, these results proved that the expanded hESCs in chitin alginate fibers could be directed to differentiate into a specific lineage, while preserving the 3D multicellular tissue-structure.

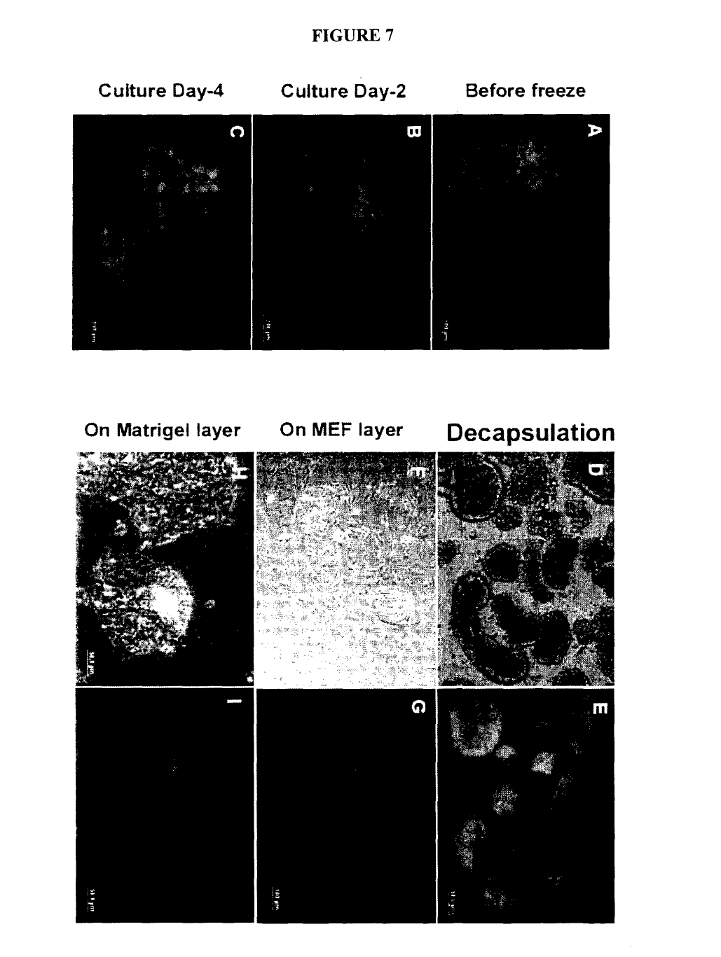

It was also tested whether hESCs encapsulated in fibers could be preserved in cell-fiber constructs while keeping self-renewal phenotype. 6-day-culture cell-fiber constructs were transferred into the ES freezing medium and preserved it following a general cell freezing procedure. After thawing, the encapsulated hESCs exhibited similar GFP fluorescent intensity compared to those before cryopreservation, and proliferated with time in culture (FIG. 7A-C). After being released from fiber, those hESCs could be easily attached on MEF layer or Matrigel-coated plates, and exhibited packed clones with similar GFP fluorescence intensity compared to unfrozen hESCs, suggesting that they maintained self-renewal phenotype (FIG. 7E-I).

In summary, an engineered 3D fibrous culture system is described that can be devised for either expansion or selective differentiation of hESCs with preservation of 3D tissue-like structure. Such an approach provides the potential for large-scale culture and separation of hESCs and their derivatives, paving the way towards producing transplantable tissue aggregates for cell-based therapy.

### REFERENCE LIST

[1] Yim E K, Wan A C. Le Visage C, Liao I C, Leong K W. Proliferation and differentiation of human mesenchymal stem cell encapsulated in polyelectrolyte complexation fibrous scaffold. Biomaterials. 2006; 27:6111-6122.
[2] Wan A C, Yim E K, Liao I C, Le Visage C, Leong K W. Encapsulation of biologics in self-assembled fibers as biostructural units for tissue engineering. J Biomed Mater Res A. 2004; 71:586-595.
[3] Sannan T, Kurita K, Iwakura Y. Studies on Chitin .2. Effect of Deacetylation on Solubility. Makromolekulare Chemie-Macromolecular Chemistry and Physics. 1976; 177:3589-3600.
[4] Xu C. Inokuma M S, Denham J et al. Feeder-free growth of undifferentiated human embryonic stem cells. Nat Biotechnol. 2001; 19:971-974.
[5] Eisen M B. Spellman P T, Brown P O, Botstein D. Cluster analysis and display of genome-wide expression patterns. Proc Natl Acad Sci USA. 1998; 95:14863-14868.
[6] Saldanha A J. Java Treeview—extensible visualization of microarray data. Bioinformatics. 2004; 20:3246-3248.

1. A method of releasably encapsulating pluripotent embryonic stem cells in a degradable continuous polyionic fiber for tissue culture, wherein the encapsulated embryonic stem cells are able to maintain a pluripotent phenotype in culture; the method comprising:
(a) contacting an aqueous solution of a polyanion with an aqueous solution of a polycation to form an interface between the aqueous solution of polyanion and the aqueous solution of polycation, and wherein the aqueous solution of polyanion or the aqueous solution of polycation or both the aqueous solution of polyanion and the aqueous solution of polycation comprises a suspension of pluripotent embryonic stem cells;
(b) drawing a continuous polyionic fiber which comprises encapsulated pluripotent embryonic stem cells from the interface; and
(c) passing the continuous polyionic fiber comprising encapsulated pluripotent embryonic stem cells in a continuous process through a solution which reduces secondary complexation of the components of the polyionic fiber,
wherein the polyanion comprises alginate and the polycation comprises chitin or chitosan.

2. The method of claim 1 further comprising culturing the encapsulated pluripotent embryonic stem cells.

3. A method of releasing encapsulated human pluripotent embryonic stem cells from a continuous polyionic fiber which comprises chitin and alginate, the method comprising exposing the polyionic fiber comprising encapsulated cells to chitinase or to chitinase and alginate lyase simultaneously and/or sequentially to degrade the polyionic fiber sufficiently to allow the release of the encapsulated cells from the polyionic fiber.

4. The method of releasably encapsulating pluripotent embryonic stem cells of claim 1, the method further comprising cryopreserving the encapsulated pluripotent embryonic stem cells.

5. The method of claim 1, wherein the cells are human pluripotent embryonic stem cells.

6. The method of claim 1, wherein the solution which reduces secondary complexation of the components of the polyionic fiber comprises CaCl₂.

7. The method of claim 3, wherein the polyionic fiber is prepared by a method of releasably encapsulating pluripotent embryonic stem cells in a degradable continuous polyionic fiber for tissue culture, wherein the encapsulated embryonic stem cells are able to maintain a pluripotent phenotype in culture; the method comprising:
(a) contacting an aqueous solution of a polyanion with an aqueous solution of a polycation to form an interface between the aqueous solution of polyanion and the aqueous solution of polycation, and wherein the aqueous solution of polyanion or the aqueous solution of polycation or both the aqueous solution of polyanion and the aqueous solution of polycation comprises a s pension of pluripotent embryonic stem cells;
(b) drawing a continuous polyionic fiber which comprises encapsulated pluripotent embryonic stem cells from the interface; and
(c) passing the continuous polyionic fiber comprising encapsulated pluripotent embryonic stem cells in a continuous process through a solution which reduces secondary complexation of the components of the polyionic fiber,
wherein the polyanion comprises alginate and the polycation comprises chitin or chitosan.

8. The method of claim 1, wherein the chitin is a water soluble chitin with a degree of deacetylation of approximately 50%.

9. An assembly for maintaining pluripotent embryonic stem cells in culture, comprising a degradable continuous polyionic fiber comprising releasably encapsulated pluripotent embryonic stem cells, wherein the polyionic fiber is held by a fiber holder, and wherein the assembly is of neutral buoyancy or is submersible in culture medium, wherein the polyionic fiber is formed from a polyanionic solution comprising alginate and a polycationic solution comprising chitin and/or chitosan.

10. The assembly of claim 9, wherein the cells are human pluripotent embryonic stem cells.

11. The assembly of claim 9, wherein the degradable continuous polyionic fiber comprising encapsulated cells is prepared by a method of releasably encapsulating pluripotent embryonic stem cells in a degradable continuous polyionic fiber for tissue culture, wherein the encapsulated embryonic stem cells are able to maintain a pluripotent phenotype in culture; the method comprising:
(a) contacting an aqueous solution of a polyanion with an aqueous solution of a polycation to form an interface between the aqueous solution of polyanion and the aqueous solution of polycation, and wherein the aqueous solution of polyanion or the aqueous solution of polycation or both the aqueous solution of polyanion and the aqueous solution of polycation comprises a suspension of pluripotent embryonic stem cells;
(b) drawing a continuous polyionic fiber which comprises encapsulated pluripotent embryonic stem cells from the interface; and
(c) passing the continuous polyionic fiber comprising encapsulated pluripotent embryonic stem cells in a continuous process through a solution which reduces secondary complexation of the components of the polyionic fiber,
wherein the polyanion comprises alginate and the polycation comprises chitin or chitosan.

12. The assembly of claim 9, wherein the chitin is a water soluble chitin with a degree of deacetylation of approximately 50%.

13. The method of claim 2, wherein the cells are human pluripotent embryonic stem cells.

14. The method of claim 4, wherein the cells are human pluripotent embryonic stem cells.

15. The method of claim 4, wherein the solution which reduces secondary complexation of the components of the polyionic fiber comprises CaCl₂.

16. The method of claim 4, wherein the chitin is a water soluble chitin with a degree of deacetylation of approximately 50%.

17. The assembly of claim 10, wherein the degradable continuous polyionic fiber comprising encapsulated cells is prepared by a method of releasably encapsulating pluripotent embryonic stem cells in a degradable continuous polyionic fiber for tissue culture, wherein the encapsulated embryonic stem cells are able to maintain a pluripotent phenotype in culture; the method comprising:
(a) contacting an aqueous solution of a polyanion with an aqueous solution of a polycation to form an interface between the aqueous solution of polyanion and the aqueous solution of polycation, and wherein the aqueous solution of polyanion or the aqueous solution of polycation or both the aqueous solution of polyanion and the aqueous solution of polycation comprises a suspension of pluripotent embryonic stem cells;
(b) drawing a continuous polyionic fiber which comprises encapsulated pluripotent embryonic stem cells from the interface; and
(c) passing the continuous polyionic fiber comprising encapsulated pluripotent embryonic stem cells in a continuous process through a solution which reduces secondary complexation of the components of the polyionic fiber,
wherein the polyanion comprises alginate and the polycation comprises chitin or chitosan.

18. The assembly of claim 17, wherein the solution which reduces secondary complexation of the components of the polyionic fiber comprises CaCl₂.

19. The assembly of claim 11, wherein the solution which reduces secondary complexation of the components of the polyionic fiber comprises CaCl₂.
